(19) United States
(12) Plant Patent Application Publication
(10) Pub. No.: US 2008/0010711 P1
(43) Pub. Date: Jan. 10, 2008

(54) Pear variety named 'Rode Doyenne Van Doorn'

(76) Inventors: Simon Klaas Broertges, Deil (NL); J.H. Van Doorn, Deil (NL)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 11/900,746

(22) Filed: Sep. 13, 2007

(30) Foreign Application Priority Data
Jan. 25, 2002 (EM) 2002/0114

(51) Int. Cl. A01H 5/00

(52) U.S. Cl. PLT/176

(57) ABSTRACT

A new pear variety is provided distinguished by its unique coloration wherein 40-60% of the fruit of the new pear variety is colored red with a green background.

### GENUS AND SPECIES OF PLANT CLAIMED

Pyrus Communis L.

1. Field of the Invention

The present invention relates to a new pear variety distinguished by its unique coloration wherein 40-60% of the fruit of the new pear variety is colored red with a green background.

2. Background of the Invention

A new distinct pear variety is described herein. This new and distinct variety named ‘Rode Doyenne Van Doorn’ resulted from a spontaneous mutation of the ‘Doyennee du Comice’ variety. The fruit of the new variety is characterized by its late season maturation and a 40-60% red coloring against a green background.

### SUMMARY OF THE INVENTION

The new variety is similar to the ‘Doyenee du Comice’ variety. The ‘Rode Doyenne Van Doorn’ variety is distinguished from other pear varieties by its distinct coloration which makes the fruits unique and offers proximate consumers a greater variety of pears from which to choose. Typically, the fruits of the standard ‘Doyennee du Comice’ variety are distinguished by their full green coloration. However, the fruits of the ‘Rode Doyenne Van Doorn’ variety, a mutation of the ‘Doyennee du Comice’, are significantly different in appearance as they have a coloration which is 40-60% red against a green background

Although the appearance of the ‘Rode Doyenne Van Doorn’ variety is different, the new variety maintains many of the same qualities as the ‘Doyenee du Comice’ such as the flavor, texture and perfume. Therefore, the quality of the fruit is not compromised for aesthetic appeal.

Characteristics of the new variety may change to adapt to different environmental conditions such as varying temperature, levels of irrigation, soil conditions and nutrients among other factors. The color descriptions and terminology used herein are used in accordance with their ordinary dictionary definitions unless indicated otherwise.

### BRIEF DESCRIPTION OF THE DRAWINGS

The accompanying photographs show typical specimens of the fruit, leaves and trees of the new variety, in color and as nearly true as reasonably possible in color illustrations of this type.

FIG. 1 is a photograph close-up view showing a single fruit of the new variety removed from its tree.

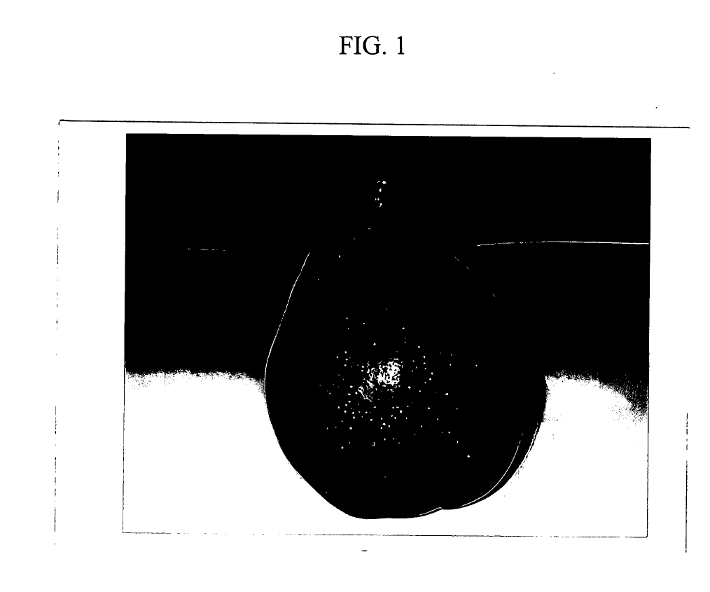

FIG. 2 is a photograph showing the fruits and leaves of the new variety.

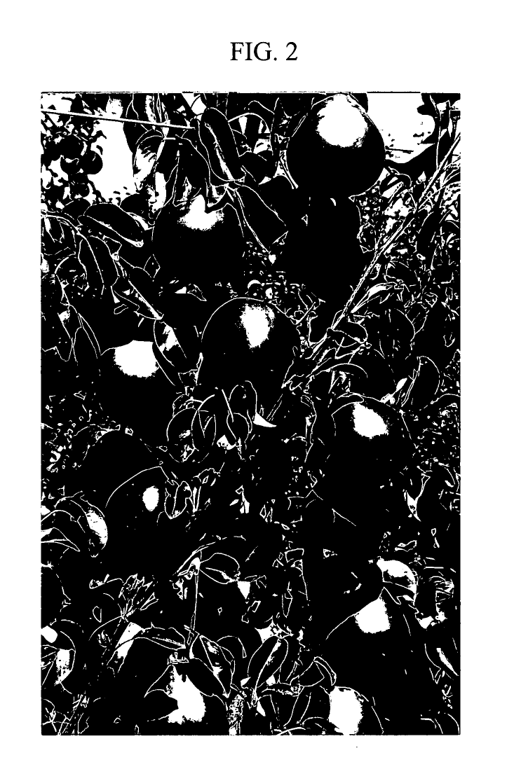

FIG. 3 is a photograph showing typical trees of the new variety.

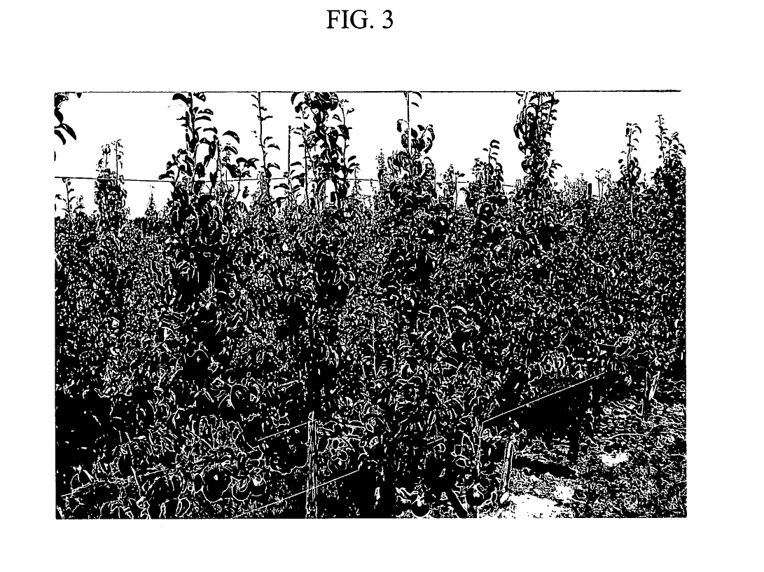

### DETAILED BOTANICAL DESCRIPTION

The following detailed description of the new pear variety, ‘Rode Doyenne Van Doorn’ is based upon observances of the plants and fruits grown and is believed to apply to the plants of the ‘Rode Doyenne Van Doorn’ grown in similar conditions. The new variety is the result of a spontaneous mutation of the ‘Doyenee du Comice’ variety and it belongs to the species Pyrus Communis L.

The new variety was discovered in Province Noord Holland in the Netherlands. Asexual propagation of the new variety maintains uniformity and stability of ‘Rode Doyenne Van Doorn.’ Asexual reproduction of the new variety was performed by the inventor by grafting and budding. The grafting and budding were performed under growing circumstances normal for pear trees with sufficient or regular water irrigation. The new variety is most clearly distinguished from the ‘Doyenee du Comice’ in that 40-60% of the ‘Rode Doyenne Van Doorn’ has a red coloring, whereas typical members of the variety are completely colored green.
Tree: the tree of ‘Rode Doyenne Van Doorn’ is similar in size to the tree of ‘Doyenee du Comice’, that is to say, upright, conical, medium in size and annually productive.
   Vigor.—medium.
   Branching.—medium.
   Habit.—upright.
   Growth (one year old shoot).—straight.
   Length of internode (one year old shoot).—medium.
   Number of lenticels (one year old shoot).—many.
   Size of lenticels (one year old shoot).—medium.
   Predominant color on sunny side (one year old shoot).—brownish red.
   Shape of apex of vegetative bud (one year old shoot).—acute.
   Position of vegetative bud in relation to shoot (one year old shoot).—markedly held out.
   Size of bud support (one year old shoot).—medium.
   Anthocyanin coloration of growing tip (young shoot; during rapid growth).—weak.
   Intensity of pubescence (young shoot; upper third).—medium.
Leaves:
   Shape.—ovoid.
   Shape of base.—truncate.
   Shape of apex.—intermediate (between acute and obtuse).
   Length.—medium.
   Width.—medium.
   Ratio of length to width.—medium.
   Margin.—few serrations.
   Incisions of margin (upper half).—absent.
   Shape of petal.—ovoid.
   Attitude in relation to shoot.—outwards.
   Curvature of longitudinal axis.—medium.
   Petiole length.—medium.
   Petiole length/leaf length ratio.—medium.
   Petiole presence of stipules.—present.
   Petiole distance of stipules from basal attachment of petiole.—short.
Flowers:
   Location of flower bud.—mainly on spurs.
   Length of flower bud.—medium.
   Sepal length.—medium.
   Attitude of sepals in relation to corolla.—spreading.
   Position of margin of petals.—touching.
   Position of stigma in relation to stamens.—same level.
   Size of petal.—medium.
   Shape of petal (excluding the claw).—broad ovate.
   Shape of base of petal (excluding the claw).—rounded.
   Length of claw of petal.—medium.
   Color of sepals.—reddish brown.
Fruits:
   Shape.—short bottle form; pyriform.
   Size.—large.
   Position of maximum diameter.—slightly towards the calyx.
   Symmetry (in longitudinal axis).—slightly asymmetric.
   Profile of sides.—straight.
   Texture of skin.—smooth.
   Sweetness.—high.
   Aroma of flesh.—rich.
   Ground color of skin.—yellow green.
   Relative area of over-color.—large.
   Hue of over-color.—dark red.
   Color of young fruit.—colored (not green).
   Ground color.—reddish yellow.
   Number of spurs.—few.
   Russet amount.—low.
   Relative area of russet around eye basin.—small.
   Relative area of russet on cheeks.—absent or very small.
   Relative area of russet around stalk attachment.—small.
   Length of stalk.—short.
   Thickness of stalk.—medium.
   Curvature of stalk.—absent or very weak.
   Attitude of stalk in relation to axis of fruit.—oblique.
   Depth of stalk cavity.—medium.
   Attitude of sepals (at harvest).—spreading.
   Eye basin (at harvest).—present.
   Depth of eye basin (at harvest).—deep.
   Width of eye basin (at harvest).—broad.
   Relief of area around eye (at harvest).—slightly ribbed.
   Firmness of flesh.—medium.
   Juiciness of flesh.—very juicy.
   Seed shape.—ovate.
   Time of beginning of flowering.—late.
   Time of maturity for consumption.—late.
   Productivity.—medium.
   Resistance to scabs.—not resistant.
   Sensitivity to scabs.—medium.

The fruits of the new variety are comparable in size to the fruits of the ‘Doyenee du Comice’ variety, which is to say that the majority of fruits produced by the new variety are large. As indicated, the fruit of the new variety is distinctly colored 40-60% deep red on a green background as opposed to the ‘Doyenee du Comice’ variety which is completely green.

Although the amount of growth and size of the fruit in any particular season depends on numerous factors including soil type, rainfall, and root type among other factors, the ‘Rode Doyenne Van Doorn’ shares similar growth habits to the standard ‘Doyenee du Comice’ when subjected to similar conditions. The ‘Rode Doyenne Van Doorn’ variety may be grown in any region in the world suitable for growing fruit trees. Normal temperate conditions are considered suitable for growing the fruits of the new variety.

1. A new and distinct variety of pear tree substantially as shown and described herein.
